(19) United States
(12) Patent Application Publication
(10) Pub. No.: US 2008/0226269 A1
(43) Pub. Date: Sep. 18, 2008

(54) Vapor generator

(75) Inventors: Donald E. DeWitt, Syracuse, IN (US); Joshua E. DeWitt, Syracuse, IN (US); T. Scott DeWitt, Syracuse, IN (US)

Correspondence Address: CARY R. REEVES, 13902 Meadowbrook Drive, Broomfield, CO, 80020, US

(73) Assignee: Innovative Instruments, Inc.

(21) Appl. No.: 11/724,743

(22) Filed: Mar. 16, 2007

(51) Int. Cl. A61L 9/03

(52) U.S. Cl. 392/386

(57) ABSTRACT

The present invention provides a vapor generator for evaporating a volatile substance into the atmosphere. The vapor generator includes a heat source, a reservoir, and a wick. In one aspect of the invention, the vapor generator output is adjustable by rotating the reservoir. In another aspect of the invention, a visual indicator indicates the relative output setting of the vapor generator. In another aspect of the invention, the reservoir is mounted relative to the heat source by magnetic attraction.

### FIELD OF THE INVENTION

The invention relates to vapor generators.

### BACKGROUND

Electrical oil vapor generators in the form of plug-in air fresheners are known in the art. A wick is placed in a bottle of fragrance oil and the fragrance evaporates from the wick to freshen the air in a room. A heater placed near the wick heats the oil and increases the rate of evaporation to increase the output of the generator. Some units have replaceable bottles to allow the user to replenish or change the fragrance oil. What is needed is a convenient and reliable way of attaching and detaching modular components such as replaceable bottles. In addition, there have been many attempts to provide a user adjustable output. These prior designs include varying the airflow over the wick, varying the heater temperature, and varying the placement of the wick relative to the heater. What is needed is a reliable user adjustable vapor generator that is cost effective to produce and simple to operate.

### SUMMARY

The present invention provides a vapor generator for evaporating a volatile substance into the atmosphere. The vapor generator includes a heat source, a reservoir containing the volatile substance, and a wick.

In one aspect of the invention, the reservoir is mounted relative to the heat source in relative rotating relationship about a rotation axis.

In another aspect of the invention a wick is mounted to the reservoir in contact with the volatile substance. The evaporative rate of the volatile substance is adjustable by rotating the reservoir relative to the heat source.

In another aspect of the invention, the vapor generator includes a wick support mounted to the reservoir and supporting the wick in a position offset radially from the rotation axis for eccentric rotation about the rotation axis. The wick and heat source define a gap between them. The wick is rotatable between a first position in which the gap is relatively smaller and a second position in which the gap is relatively larger.

In another aspect of the invention, the vapor generator includes a heat shield covering a circumferential portion of the wick. The heat shield is rotatable between a first position in which more of the heat shield is interposed between the wick and the heat source and a second position in which less of the heat shield is interposed between the wick and the heat source.

In another aspect of the invention, the wick has a non-circular cross sectional shape. The wick and heat source define a gap between them. The wick is rotatable between a first position in which the gap is relatively smaller and a second position in which the gap is relatively larger.

In another aspect of the invention, the vapor generator includes a base including a magnetic material and the reservoir includes a magnetic material. The reservoir is mounted to the base by magnetic attraction.

In another aspect of the invention, a method includes providing a vapor generator including a heater and a reservoir assembly including a reservoir and wick; mounting the reservoir assembly for rotation relative to the heater about a rotation axis; rotating the reservoir assembly about the rotation axis to vary the output of the vapor generator.

### BRIEF DESCRIPTION OF THE DRAWINGS

Various examples of the present invention will be discussed with reference to the appended drawings. These drawings depict only illustrative examples of the invention and are not to be considered limiting of its scope.

FIG. 1 is a perspective view of a vapor generator according to the present invention;

FIG. 2 is a perspective view of a reservoir assembly for use with the generator of FIG. 1;

FIG. 3 is an exploded perspective view of the generator of FIG. 1;

FIG. 4 is a top view of the reservoir assembly of FIG. 2;

FIG. 5 is a is a perspective section view of the generator of FIG. 1;

FIG. 6 is a top view of the generator of FIG. 1 in a first operative state;

FIG. 7 is a top view of the generator of FIG. 1 in a second operative state;

FIG. 8 is a perspective view of a reservoir assembly for use with the generator of FIG. 1;

FIG. 9 is a perspective view of a reservoir assembly for use with the generator of FIG. 1;

FIG. 10 is a perspective view of a reservoir assembly for use with the generator of FIG. 1; and

FIG. 11 is a top plan view of the reservoir assembly of FIG. 10.

### DESCRIPTION OF THE ILLUSTRATIVE EXAMPLES

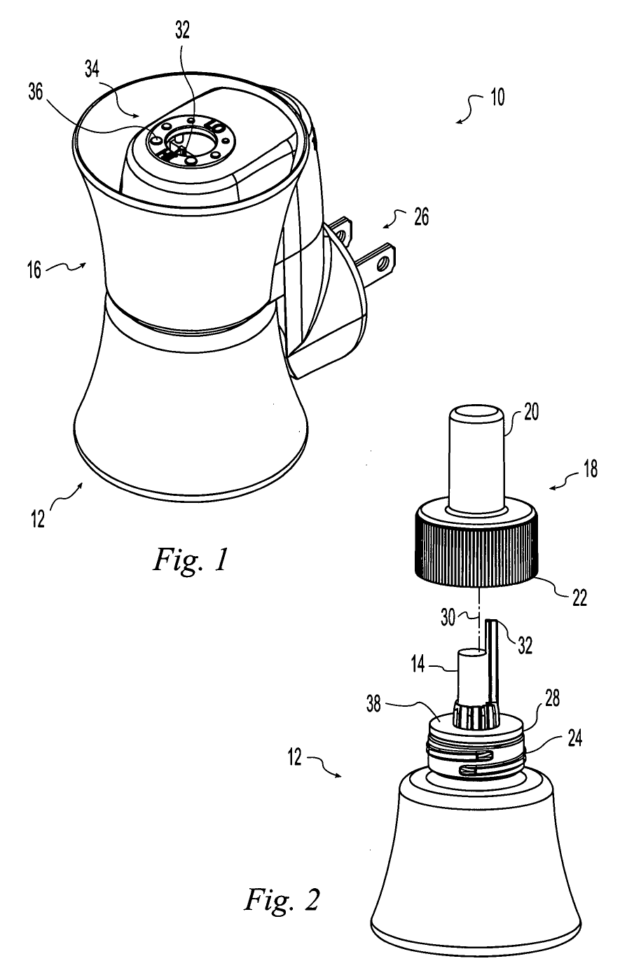

Referring to FIGS. 1-2, a vapor generator 10 includes a reservoir assembly 12, including a volatile substance and a wick 14, and a heater assembly 16. The volatile substance may include a pharmaceutical, a fragrance, an odor eliminator, an insecticide, a repellent and/or any other substance that is desirable to release into the environment. For example, the volatile substance may include a fragrance oil. The reservoir assembly 12 may be a modular unit able to be changed during manufacture and/or by a user, an integral unit fillable during manufacture and/or by a user, and/or any other suitable reservoir assembly of any shape or size. For example, the reservoir assembly 12 may include a modular unit changeable by the user to provide variety of volatile substances and/or to refill the generator 10. The reservoir assembly 12 may be supplied with a cap 18 for preventing vaporization of the volatile substance and spillage when the reservoir assembly is not in use with the heater assembly 16. For example, a cap 18 may include an enclosed wick cover 20 and a threaded portion 22 engageable with a threaded periphery 24 on the reservoir assembly 12 to cover the wick 14 and seal in the volatile substance. The wick 14 may be a fibrous structure, a monolithic porous structure, a tubular structure, and/or any other suitable structure for conducting the volatile substance from the reservoir assembly to the atmosphere. For example, the wick 14 may include a porous polymer structure. The heater assembly 16 may include an electrical heater, a chemical heater, a combustion heater, and/or any other suitable heater. An electrical heater may be powered by batteries, A/C outlet, and/or other suitable power supply. For example, the heater assembly 16 may include a plug 26 adapted for insertion into an A/C outlet.

The reservoir assembly 12 may include a heater assembly engagement portion 28 engageable with the heater assembly 16 for rotation about an axis 30. Rotation of the reservoir assembly 12 may provide for orientation of decorative elements in a user desirable orientation, output control, and/or any other feature for which a rotatable reservoir assembly 12 may be desirable.

The wick 14 may be offset from the rotation axis 30 so that the wick 14 moves eccentrically about the rotation axis 30 as the wick and/or reservoir assembly is rotated about the rotation axis 30. As the reservoir assembly 12 is rotated about the rotation axis 30, the wick 14 changes position relative to the heater assembly 16. Different positions result in varying heating of the wick 14 and thus varying evaporative rates of the volatile substance. The reservoir assembly 12 may engage the heater assembly 16 for rotation through a few degrees, many degrees, or continuous rotation. Rotation of at least 180 degrees, while not necessary, provides the broadest range of eccentric placement of the wick 14 relative to the heater assembly 16. Continuous rotation results in continuous adjustability from maximum output to minimum output and back to maximum output in one revolution of the reservoir assembly 12 about the rotation axis 30.

The wick 14 may have a portion covered by a heat shield such that rotation of the wick, heat shield, and/or reservoir assembly varies the heat absorbed by the wick and thus the evaporative rate of the volatile substance.

The wick 14 may have a non-circular cross sectional shape such that rotation of the wick and/or reservoir assembly varies the heat absorbed by the wick and thus the evaporative rate of the volatile substance.

The reservoir assembly may also include an indicator 32 for indicating the position of the wick 14 relative to the heater assembly 16. The indicator 32 may be visible from the top of the generator 10 to simplify checking the wick 14 position by a user looking down on the generator 10. For example, the indicator 32 may be offset from the rotation axis 30 so that it moves eccentrically with the wick 14 and indicates the wick 14 position. The indicator 32 may be readable adjacent to a portion 34 of the top of the generator 10 to indicate the relative output of the generator 10. The portion 34 may include indicia 36 indicating the relative generator 10 output. As the reservoir assembly 12 is rotated about the rotation axis 30, the indicator 32 may rotate to be adjacent different indicia 36. The indicator 32 may be offset in the same direction as the wick 14 or in any other direction relative to the rotation axis 30. For example, the indicator 32 may be offset opposite the wick 14 so that the indicator 32 and wick 14 rotate eccentrically about the rotation axis 30 in opposed relationship as shown in FIG. 2.

The reservoir assembly 12 may attach to the heater assembly 16 by threads, snap-fit, press-fit, magnetic attraction, and/or any other mechanism. For example, the reservoir assembly 12 may include a magnetic material 38 that is attracted to another magnetic material included in the heater assembly 16. The magnetic attraction of the magnetic material 38 may provide simplified removal and replacement of the reservoir assembly 12. The magnetic attraction of the magnetic material 38 may provide for rotation of the reservoir assembly 12 relative to the heater assembly 16. For example, the magnetic material 38 may provide for continuous non-binding rotation of the reservoir assembly 12 relative to the heater assembly 16.

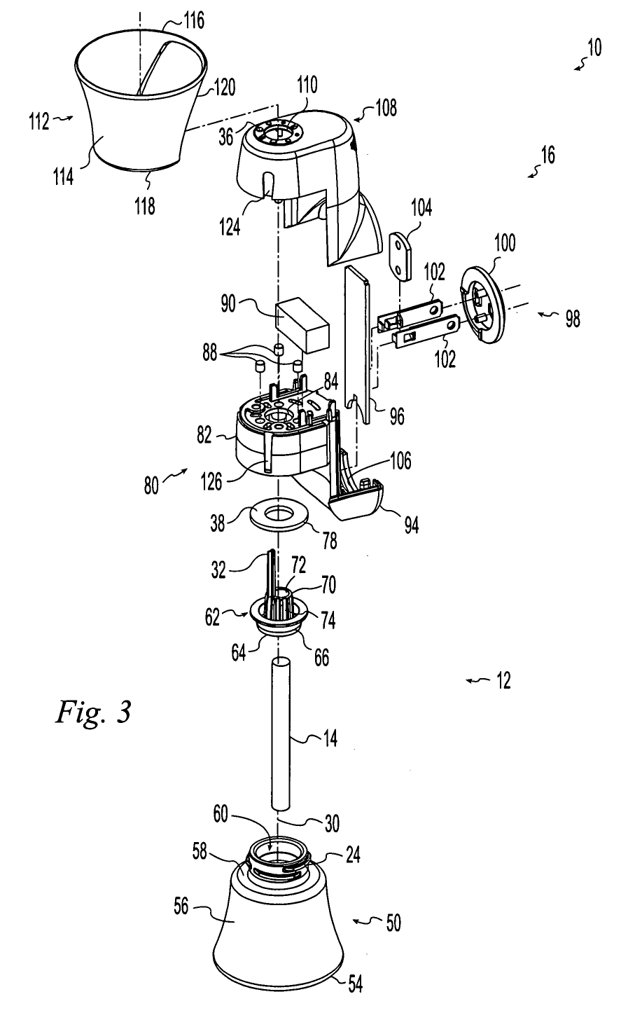

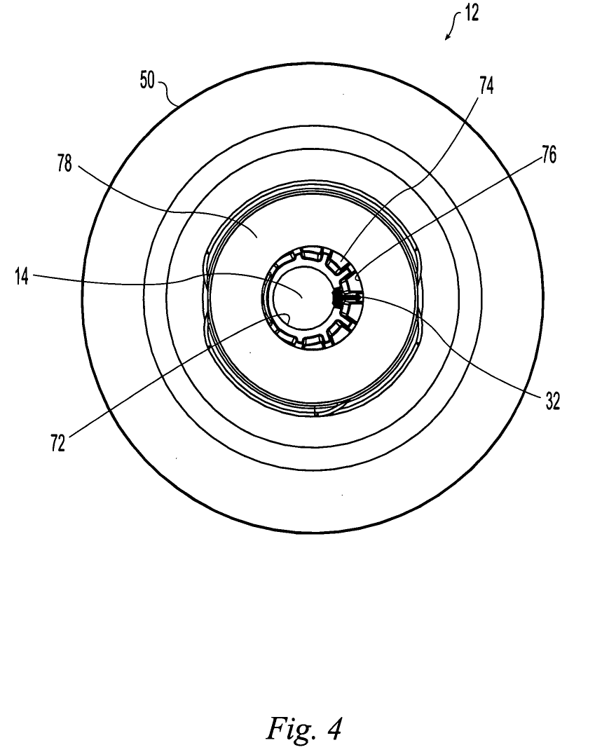

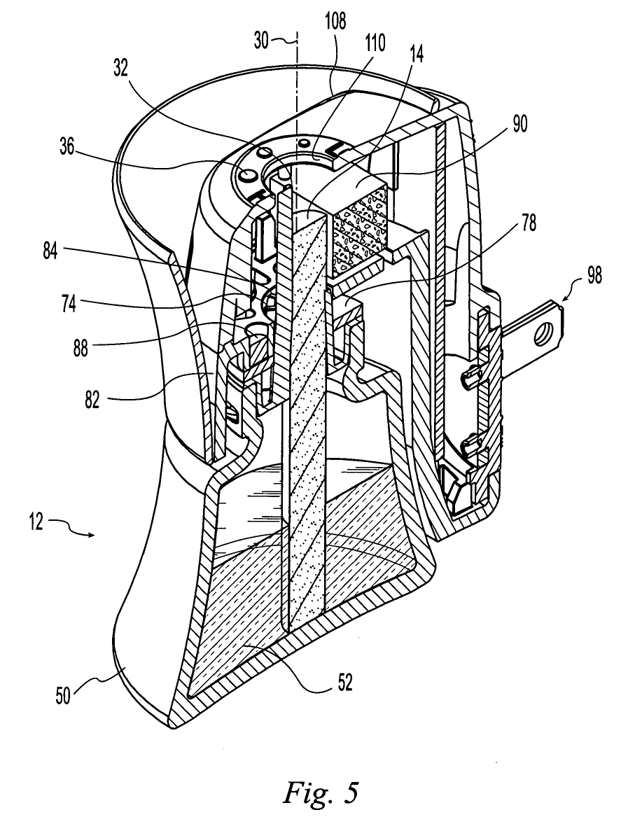

Referring now to FIGS. 3-5, an exemplary embodiment of a vapor generator 10 is shown in detail. The reservoir assembly 12 includes a reservoir 50 able to contain a volatile substance 52 (FIG. 5). The reservoir 50 includes a bottom wall 54, a continuous sidewall 56, and a top 58 defining an opening 60. The top 58 includes a threaded periphery 24 to releasably receive the storage cap 18 of FIG. 2. A reservoir plug 62 includes a bottom 64 defining an annular seat 66 that forms a press-fit with the opening 60 of the reservoir 50. A generally tubular wick support 70 extends upwardly from the seat 66 and defines a through bore 72 communicating with the interior of the reservoir 50. A plurality of fins 74 buttress the wick support 70 and extend radially outwardly from it. The radial outer limits of the fins 74 define a circle 76 (FIG. 4) the center of which lies on the rotation axis 30 of the reservoir assembly 12. The through bore 72 is offset laterally from the rotation axis 30 and is parallel to the rotation axis 30. One of the fins 74 extends upwardly beyond the other fins to define the indicator 32. The wick 14 is received through the through bore 72 of the wick support 70 and the plug 62 is pressed into the opening 60 of the reservoir 50. A magnetic washer 78 is pressed over the fins 74 to provide the magnetic material 38 adjacent the top of the reservoir assembly 12. The reservoir assembly is shown in top view in FIG. 4. Note that the wick 14 is offset to the left and the indicator 32 is offset to the right in FIG. 4 so that they move about the rotation axis 30 in opposed eccentric relationship.

Continuing with FIG. 3, a heater assembly 16 includes a lower housing 80 including a reservoir assembly receiving base 82. The base defines an opening 84 able to receive the fins 74 in axial rotating relationship. The base further defines blind openings 86 receiving magnets 88 for attracting the washer 78 to attach the reservoir assembly to the base 82. The washer 78 bears on the bottom of the base 82 and provides for smooth continuous rotation. A resistance heater 90 is positioned by clips 92 adjacent to the opening 84.

The lower housing 80 includes a plug portion 94 depending from the base 82. A circuit board 96 is mounted to the plug portion 94 and the heater 90 is connected to the circuit board 96 with electrical conductors (not shown). A plug assembly 98 includes a terminal disk 100, a pair of terminals 102 extending through the terminal disk 100, and a terminal disk plate 104 connected to the terminal disk 100, such as by heat staking or some other suitable method, to trap the terminals 102 on the terminal disk 100. The plug assembly 98 is mounted to the plug portion 94 of the lower housing 80 with the terminal disk 100 received in a groove 106 for rotation. The plug assembly 98 can thus rotate through at least 90 degrees to accommodate both vertical and horizontal electrical outlets. The terminals 102 are connected to the circuit board 96 in rotating electrical relationship. Traces on the circuit board 96 connect the terminals 102 to the heater 90 so that when the plug assembly 98 is inserted into an electrical outlet, electricity is conducted to the heater 90 and the heater 90 generates heat. An upper housing 108 mounts to the lower housing 80 and encloses magnets 88, heater 90, circuit board 96, and plug assembly 98. The upper housing 108 can be connected to the lower housing 80 by adhering, staking, snapping, and/or by any other suitable method. The upper housing 108 defines an opening 110 aligned with the opening 84 in the lower housing 80. The opening 110 has a diameter sufficiently large to receive the indicator 32 and permit it to rotate through its predefined range of motion about the rotation axis 30. Indicia 36 are formed on the top of the upper housing 108 adjacent to the opening 110. The position of the indicator 32 adjacent to the indicia 36 indicates, by reading the indicated indicia, the relative output of the vapor generator 10.

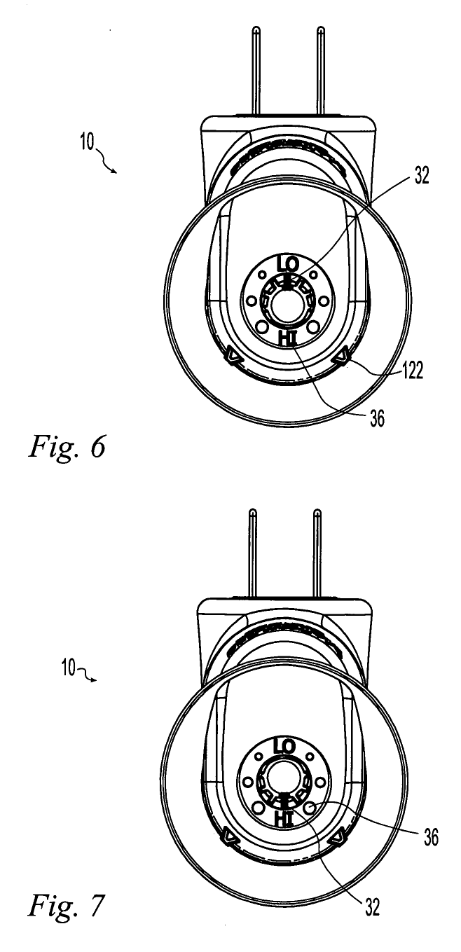

A shade 112 includes a generally frustoconical side wall 114 open at the top 116, bottom 118, and one side 120. The shade 112 fits over the upper housing 108 to form a decorative cover. The shade 112 includes tabs 122 (FIG. 6) that snap into grooves 124, 126 in the upper and lower housings 108, 80. The shade 112 may include figures, scenes, patterns, and/or other depictions molded into it or applied to it to vary its appearance. It may be made of different materials such as plastic, paper, glass, opaque materials, translucent materials, and/or other suitable materials to achieve a desired visual effect. The different shades may be used in manufacturing and/or provided to the consumer for customization of the vapor generator 10. For example, various seasonal themes may be printed on the shade 112 and provided to the consumer to allow end user to customize of the look of the vapor generator 10.

The operation of the illustrative vapor generator 10 is apparent from FIG. 5. A user selects a reservoir assembly 12 containing a desirable volatile substance 52 and attaches it to the base 82 by inserting it through the bottom of the lower housing 80. The indicator 32, wick 14, and fins 74 extend through the opening 84 in the base 82 until the indicator 32 is adjacent the indicia 36 on the upper housing 108 and the wick 14 is positioned near the heater 90. The magnets 88 attract the washer 78 and hold it against the bottom of the base 82 in continuously rotatable relationship. The plug assembly 98 is connected to a power source and the heater 90 begins to heat the wick 14. The volatile substance 52 is drawn up through the wick 14 and evaporates from the top of the wick 14 such that vapors of the volatile substance 52 are emitted through the opening 110 in the upper housing 108. In the position shown in FIG. 5, the wick 14 is at its closest spacing from the heater 90 so that the wick 14 is heated at the maximum rate and the volatile substance 52 evaporates at the maximum evaporative rate. In this position, the indicator 32 and indicia 36 indicate the high output setting. To adjust the evaporative rate, and thus the output from the vapor generator 10, a user grasps the reservoir assembly 12 by the reservoir 50 and rotates it about the rotation axis 30. As the reservoir assembly 12 rotates, the wick 14 moves eccentrically away from the heater 90. The further the wick 14 is positioned from the heater 90 the lower the output from the vapor generator 10. At 180 degrees of rotation, the wick 14 is at its furthest spacing from the heater 90 and so that that the wick is heated at the minimum rate and the volatile substance 52 evaporates at the minimum evaporative rate. The output is continuously adjustable between the maximum and minimum settings. Indicia 36 are provided to allow a user to repeatably dial in a desired output. Continued rotation beyond 180 degrees begins to increase the output until at 360 degrees of rotation the output is again at a maximum.

FIGS. 6 and 7 illustrate the vapor generator 10 from the top as it would be seen by a user looking down on it, for example, when it is plugged into an electrical outlet below eye level. The indicator 32 is clearly visible from the top to allow checking of the wick 14 setting at a glance. The indicia 36 are also visible from the top. However, even if the indicia are too small to read or are omitted, the indicator 32 is visible and indicates the setting. Like the minute hand on an analog clock face with which all users are familiar, the indicator gives an instant indication of wick position. FIG. 6 illustrates the low setting while FIG. 7 illustrates the high setting.

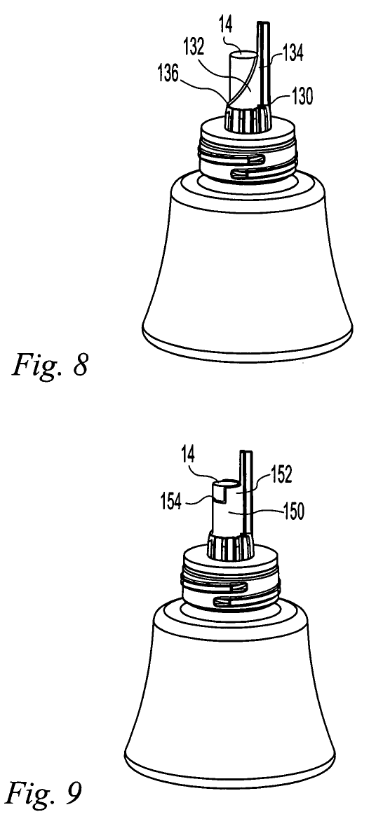

FIG. 8 illustrates an alternative way of varying the vapor generator 10 output by rotation of the reservoir assembly. In this embodiment, the wick support 130 includes a heat shield 132 enclosing a portion of the wick 14. The heat shield 132 varies from extensive shielding of the wick 14 on a first side 134 to minimal shielding of the wick 14 on a second side 136. In the illustrative embodiment, the heat shield 132 tapers from covering the entire wick 14 on the first side 134 to no coverage on the second side 136 at a location opposite the first side 134. The heat shield 132 can be shaped to provide a continuous linearly variable change in evaporative rate, a continuous non-linear change, a stepped change, and/or any other desirable change in evaporative rate responsive to rotation of the reservoir assembly.

As an alternative example to the heat shield of FIG. 8, FIG. 9 illustrates a stepped heat shield 150 having a first portion 152 that completely shields the wick and a second portion 154 that abruptly, or stepwise, exposes a portion of the wick 14. In the illustrative example, the heat shield 150 comprises a hollow cylinder surrounding the wick 14. The first portion 152 extends to the top of the wick 14 and encloses approximately 180 degrees of the wick 14. In the second portion 154, the heat shield 150 is partially removed by notching it straight down to expose approximately 180 degrees of the wick 14.

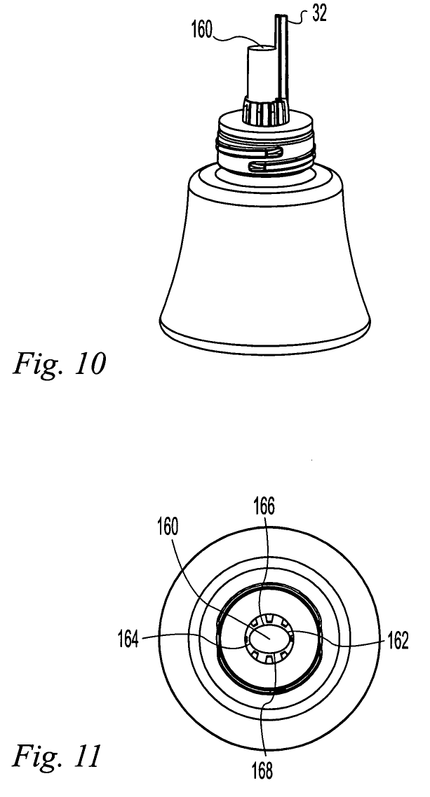

FIGS. 10 and 11 illustrate another way of varying the vapor generator 10 output by rotation of the reservoir assembly. In this embodiment, the wick 160 has a non-circular cross-sectional shape such that as the reservoir assembly is rotated, a portion of the wick is spaced nearer to or farther from the heater 90. In the illustrative example, the wick 160 has an elliptical cross-sectional shape. If the center of the wick 160 is concentric with the axis of rotation 30 of the reservoir assembly, it will produce a maximum evaporative rate when one of the sides 162, 164 corresponding to the major axis is oriented toward the heater 90 and a minimum evaporative rate when one of the sides 166, 168 corresponding to the minor axis is oriented toward the heater 90. The evaporative rate is continuously adjustable between the minimum and maximum settings. While the alternative wick 160 is shown with an elliptical shape, it may have any non-circular cross-sectional shape that will be responsive to rotation to cause a portion of the wick 160 to be nearer to or farther from the heater 90.

The embodiments of FIGS. 8-11 may have the center of the wick 14, 160 coincident with the axis of rotation of the reservoir assembly relative to the heater since they do not depend on eccentricity to vary the heat absorbed by the wick 14, 160. However, these embodiments may also be combined in any combination of eccentricity, shielding, and/or non-circular wick shape to produce a desired variable evaporative rate responsive to rotation of the reservoir assembly.

Although examples of a vapor generator and its use have been described and illustrated in detail, it is to be understood that the same is intended by way of illustration and example only and is not to be taken by way of limitation. Accordingly, variations in and modifications to the vapor generator and its use will be apparent to those of ordinary skill in the art, and the following claims are intended to cover all such modifications and equivalents.

What is claimed is:

1. A vapor generator for evaporating a volatile substance into an environment, the vapor generator comprising:
a heat source;
a reservoir containing the volatile substance, the reservoir being mounted relative to the heat source in relative rotating relationship about a rotation axis.

2. The vapor generator of claim 1 further comprising:
a wick mounted to the reservoir in contact with the volatile substance; and
means for varying the evaporative rate of the volatile substance from the vapor generator responsive to rotation of the reservoir relative to the heat source.

3. The vapor generator of claim 2 wherein the means for varying the evaporative rate comprises a wick support mounted to the reservoir, the wick support supporting the wick in a position offset radially from the rotation axis for eccentric rotation about the rotation axis, the wick and heat source defining a gap between them, the wick being rotatable between a first position in which the gap is relatively smaller and a second position in which the gap is relatively larger.

4. The vapor generator of claim 2 wherein the means for varying the evaporative rate comprises a heat shield covering a circumferential portion of the wick, the heat shield being rotatable between a first position in which more of the heat shield is interposed between the wick and the heat source and a second position in which less of the heat shield is interposed between the wick and the heat source.

5. The vapor generator of claim 4 wherein the amount of heat shield interposed between the wick and heat source varies gradually between the first and second positions.

6. The vapor generator of claim 4 wherein the amount of heat shield interposed between the wick and heat source varies abruptly in step-wise fashion between the first and second positions.

7. The vapor generator of claim 2 wherein the wick has a non-circular cross sectional shape, the wick and heat source defining a gap between them, the wick being rotatable between a first position in which the gap is relatively smaller and a second position in which the gap is relatively larger.

8. The vapor generator of claim 2 further comprising a visual indicator mounted in fixed position relative to the wick, the visual indicator being responsive to rotation of the reservoir to indicate the wick position relative to the heat source.

9. The vapor generator of claim 8 wherein the visual indicator is viewable from above the vapor generator.

10. The vapor generator of claim 9 further comprising a housing having a hole in the top, the visual indicator extending upwardly from the wick to a position adjacent the hole, the circumferential position of the visual indicator relative to the hole indicating the wick position relative to the heat source.

11. The vapor generator of claim 8 further comprising indicia formed on the housing in fixed position relative to the heat source, the position of the visual indicator relative to the indicia indicating a relative evaporative rate of the volatile substance.

12. The vapor generator of claim 1 further comprising a base, the heat source being mounted to the base, the base comprising a magnetic material and the reservoir comprising a magnetic material, the reservoir being mounted to the base by magnetic attraction.

13. A vapor generator for evaporating a volatile substance into an environment, the vapor generator comprising:
a base;
a heat source mounted to the base;
a reservoir mounted to the base in rotating relationship relative to the heat source about a rotation axis; and
a wick mounted to the reservoir, the reservoir being rotatable between a first position in which the evaporative rate of the volatile substance is relatively high and a second position in which the evaporative rate of the volatile substance is relatively low.

14. The vapor generator of claim 13 further comprising a wick support mounted to the reservoir and defining a through hole receiving the wick in a position offset from the rotation axis, the wick moving eccentrically about the rotation axis as the reservoir is rotated about the rotation axis.

15. The vapor generator of claim 13 further comprising a housing and a visual indicator, the housing having a top portion viewable from above, the visual indicator extending upwardly from the wick support and being readable relative to the top portion of the housing from above the vapor generator to indicate the position of the wick relative to the heat source.

16. The vapor generator of claim 13 wherein the base further comprises a magnetic material and the reservoir further comprises a magnetic material, the reservoir being mounted to the base by magnetic attraction.

17. The vapor generator of claim 13 further comprising a heat shield covering a circumferential portion of the wick, the heat shield being rotatable between a first position in which more of the heat shield is interposed between the wick and the heat source and a second position in which less of the heat shield is interposed between the wick and the heat source.

18. The vapor generator of claim 13 wherein the wick has a non-circular cross sectional shape, the wick and heat source defining a gap between them, the wick being rotatable between a first position in which the gap is relatively smaller and a second position in which the gap is relatively larger.

19. A vapor generator for evaporating a volatile substance into an environment, the vapor generator comprising:
a base comprising a magnetic material;
a heat source mounted to the base;
a reservoir comprising a magnetic material, the reservoir being mounted to the base by magnetic attraction; and
a wick mounted to the reservoir.

20. A reservoir assembly for use with a vapor generator for evaporating a volatile substance into an environment, the vapor generator including a heat source and a reservoir receiving portion able to engage the reservoir for rotation about a rotation axis, the reservoir assembly comprising:
a reservoir engageable with the reservoir assembly for rotation about the rotation axis;
a wick mounted to the reservoir in a position offset from the rotation axis, the wick being responsive to rotation of the reservoir about the rotation axis to move eccentrically about the rotation axis between a first position in which the evaporative rate of the volatile substance is relatively high and a second position in which the evaporative rate of the volatile substance is relatively low.

21. A reservoir assembly for use with a vapor generator for evaporating a volatile substance into an environment, the vapor generator including a heat source and a reservoir receiving portion able to engage the reservoir for rotation about a rotation axis, the reservoir assembly comprising:
a reservoir engageable with the reservoir assembly;
a wick mounted to the reservoir;
a heat shield covering a circumferential portion of the wick, the heat shield being rotatable between a first position in which more of the heat shield is interposed between the wick and the heat source and a second position in which less of the heat shield is interposed between the wick and the heat source.

22. A reservoir assembly for use with a vapor generator for evaporating a volatile substance into an environment, the vapor generator including a heat source and a reservoir receiving portion able to engage the reservoir for rotation about a rotation axis, the reservoir assembly comprising:
a reservoir engageable with the reservoir assembly;
a wick mounted to the reservoir, the wick having a non-circular cross sectional shape, the wick and heat source defining a gap between them, the wick being rotatable between a first position in which the gap is relatively smaller and a second position in which the gap is relatively larger.

23. A reservoir assembly for use with a vapor generator for evaporating a volatile substance into an environment, the vapor generator including a heat source, a reservoir receiving portion, and a magnetic material, the reservoir assembly comprising:
a reservoir including a magnetic material, the reservoir being mountable to the vapor generator by magnetic attraction; and
a wick mounted to the reservoir.

24. A method of varying the output of a vapor generator, the method comprising:
providing a vapor generator including a heater and a reservoir assembly including a reservoir and wick;
mounting the reservoir assembly for rotation relative to the heater about a rotation axis;
rotating the reservoir assembly about the rotation axis to vary the output of the vapor generator.
